(19) United States
(12) Patent Application Publication
(10) Pub. No.: US 2015/0182448 A1
(43) Pub. Date: Jul. 2, 2015

(54) TRADITIONAL CHINESE MEDICINE COMPOSITION FOR DISPELLING WRINGLES AND BLACK EYE AND ITS PREPARATION METHOD

(71) Applicant: Yuan Yuan, Beijing (CN)

(72) Inventor: Yuan Yuan, Beijing (CN)

(73) Assignee: Shang Chen Mei Bang (Beijing) Biotechnology Co., Ltd., Beijing (CN)

(21) Appl. No.: 14/144,567

(22) Filed: Dec. 31, 2013

(51) Int. Cl. A61K 8/97; A61Q 19/08; A61K 8/73; A61K 8/60; A61K 8/64

(52) U.S. Cl. CPC A61K 8/975; A61K 8/97; A61K 8/602; A61K 8/64; A61K 8/73; A61Q 19/08

(57) ABSTRACT

The invention provides a composition and method for preparing medicine for eye bags and dark circles fade, wherein said composition comprises the following parts by weight ratio of raw material composition: 10-15 parts water, Ganoderma lucidum extract 2-3 copies, black Ganoderma extract 3-4, 5-6 parts wheat germ oil, sucrose laurate 2-3 parts, 3-5 parts chamomile extract, 3-5 parts of milk protein, yeast ferment filtrate 3-5 copies, dextran 2-3 copies, copies Tremella 2-4, 2-3 parts angelica root extract, astragalus extract 2-3 parts. The above described method for preparing a composition according to the present invention, without any side effects, can be soft smooth skin around the eyes, remove free radicals, enhance the resilience of the skin, thereby improving eye skin color, to get rid of dark circles effect.

### CROSS-REFERENCE TO RELATED APPLICATIONS

This application claims the priority benefit of Chinese Patent Application Serial No. 201310703312.4, filed on Dec. 19, 2013; Chinese Patent Application Serial No. 201310685138.5, filed on Dec. 13, 2013; Chinese Patent Application Serial No. 201310687223.5, filed on Dec. 13, 2013; Chinese Patent Application Serial No. 201310685115.4, filed on Dec. 13, 2013; Chinese Patent Application Serial No. 201310684837.8, filed on Dec. 13, 2013; Chinese Patent Application Serial No. 201310685112.0, filed on Dec. 13, 2013; Chinese Patent Application Serial No. 201310686861.5, filed on Dec. 13, 2013; Chinese Patent Application Serial No. 201310687218.4, filed on Dec. 13, 2013; Chinese Patent Application Serial No. 201310686865.3, filed on Dec. 13, 2013; Chinese Patent Application Serial No. 201310687250.2, filed on Dec. 13, 2013; Chinese Patent Application Serial No. 201310684846.7, filed on Dec. 13, 2013; Chinese Patent Application Serial No. 201310685166.7, filed on Dec. 13, 2013; and Chinese Patent Application Serial No. 201310687246.6, filed on Dec. 16, 2013, the disclosure of all of which are hereby incorporated by reference in their entirety.

### FIELD OF THE INVENTION

The invention relates to a traditional Chinese medicine composition for dispelling wrinkles and black eye and its preparation method, belongs to a traditional Chinese medicine composition technology field.

### BACKGROUND OF THE INVENTION

With the accelerated pace of life, dark circles more and more people, and safe and effective to get rid of dark circles cosmetics market demand. Currently there are a lot of various eye cream patent, however, a variety of functions in one set, and highly targeted no.

The present invention yeast extract rich in vitamin PP, can enhance the recovery of capillaries in the skin, by promoting the synthesis of vascular endothelial growth factor (VEGF), the formation of new blood vessels develop. By dredge eye skin blood circulation, thereby preventing the formation of blocking vascular congestion dark circles and bags under the eyes.

### SUMMARY OF THE INVENTION

The present invention can be soft smooth skin around the eyes, the skin moist height, eye bags and dark circles fade, reduce wrinkles, crow's feet forming resist daytime pollution, anti-free radicals.

Techniques of the present invention are: the kind of traditional Chinese medicine composition for eye bags and dark circles fade, wherein said composition comprises the following parts by weight ratio of raw material composition: 10-15 parts water, Ganoderma lucidum extract object 2-3 copies, black Ganoderma extract 3-4, 5-6 parts wheat germ oil, sucrose laurate 2-3 parts, 3-5 parts chamomile extract, 3-5 parts of milk protein, yeast ferment filtrate 3-5 parts, dextran 2-3 parts, Tremella 2-4 copies, angelica root extract 2-3 parts, astragalus extract 2-3 parts.

Wherein each component may be: 10-11 parts water, 2-3 parts of the extract of Ganoderma lucidum, Ganoderma lucidum extract black parts 3-4, 5-6 parts of wheat germ oil, 2-3 parts of sucrose laurate, chamomile extract 3-4 parts, milk protein 3-4, yeast ferment filtrate 3-4, 2-3 glucan copies, copies Tremella 2-3, 2-3 parts angelica root extract, astragalus extract object 2-3 copies.

Wherein each component may be: 12-13 parts of water, 2-3 parts of the extract of Ganoderma lucidum, Ganoderma lucidum extract black parts 3-4, 5-6 parts of wheat germ oil, 2-3 parts of sucrose laurate, chamomile extract 4-5 copies, 4-5 parts of milk protein, yeast ferment filtrate 4-5 parts, dextran 2-3 parts, Tremella 3-4, 2-3 parts angelica root extract, astragalus extract object 2-3 copies.

Preparation of Chinese medicinal composition of the present invention are: (a) were weighed according to the amount of each raw material; (2) mixing the composition in water and soaked in 0.5 to 2 hours, heated to 80˜100, for 0.5 to 1.5 hrs; (3) to said step (2) for coarse filtration; (4) cooling the filtrate from step 3 to 30˜50, and vacuum filtration, the filtrate was taken to give the active extract .

Step 3 wherein said coarse filter is a gauze with 200 to 300 mesh filter. Wherein said step 4 with the vacuum suction plate and frame filter, a vacuum degree of 0.05˜0.1 Mpa, the fineness of the filtration plate 1 to 5 microns. The method can be preferably used for preparing the raw extract of the active ingredient in the drug. The components are extracted through conventional methods together with accessories made of cream.

Also provides a composition for use of the medicine, sooner or later, apply lotion or cream eye after regeneration, Apply on the skin near the eye and shake gently to help shake head penetration.

Pharmacological effects:

Ganoderma lucidum: To basidiomycete Polyporaceae Ganoderma, fruiting bodies of Ganoderma lucidum or growth in East Asia. Toxic side effects, can Edible. Black fungus, also known as black Chi, Chi alias mysterious, dark turf, fake fungus, sweet and flat. Black Chi kidney, diuretic, through Jiuqiao, Satoshi police, consumer product; Indications of acute nephritis, chronic nephritis, indigestion.

Wheat germ oil: a little malt as raw materials of a cereal germ oil, which focused on the nutritional essence of wheat, rich in vitamin E, linoleic acid, linolenic acid, sweet eight carbon alcohol and a variety of physiologically active components, is a valuable functional foods with high nutritional value.

Chamomile : Asteraceae, native to Europe. The effect of sleep to help ease the inflammation and pain of patients, relieve insomnia, itchy skin caused by the nerve of.

Yeast: Single-celled fungus, is the history of human civilization is the earliest applications of microorganisms. Can survive in anoxic environments.

Glucan: A polysaccharide. Present in the growth of certain microorganisms in the mucus secreted.

Tremella: basidiomycetes polysaccharide immune enhancer, improve immune function and increase the role of white blood cells.

Astragalus, also known as cotton Qi, with enhanced immune function, liver, diuretic, anti-aging, anti-stress, blood pressure and wider antibacterial effect.

After the composition of the present invention, the above ingredients, a synergistic effect of its efficacy.

The beneficial effects of the invention:

After testing, the invention itself herbal extracts inhibit thrombin activity and the effect of inhibiting tyrosinase activity, improve blood circulation and inhibit the formation of melanin, In addition, the detection of red blood cell hemolysis experiments no significant hemolysis. Experiments show that the gel formulations of the present invention uses a month later, the test area and the red pigment melanin levels were decreased on average, and the delicate skin of the eye without stimulation; cream of the invention a month later, the skin test area the increase in brightness. Therefore, the efficacy of the plant can be used as an additive to add it to the cosmetics, especially in the preparation of the skin due to poor microcirculation and hyperpigmentation caused by dark circles, dull skin and other skin care products in the best results.

### DETAILED DESCRIPTION OF THE PREFERRED EMBODIMENT

To further illustrate the present invention, the following examples illustrate specific example of combining:

### Example 1

Chinese medicine composition for eye bags and dark circles fade, preparation APIs are:

10 g of water, 2 g of Ganoderma lucidum extract, ganoderma extract 3 g of black, 5 grams of wheat germ oil, 2 g of sucrose laurate, chamomile extract 3 g, 3 g of milk protein, 3 g yeast ferment filtrate, dextran 2 grams of sugar, white fungus polysaccharides 2 g, 2 g angelica root extract, astragalus extract 2 g.

Preparation Method:

(1) in accordance with the amount of raw materials were weighed each drug;

(2) The above composition is immersed in water for 0.5 hours and heated to 80, incubated 40 minutes;

(3) to said step (2) with a coarse filter gauze of 250 mesh, and the combined filtrate;

(4) The step (3) of the filtrate was cooled to 50, vacuum-suction frame, the degree of vacuum is 0.5 Mpa, the filter plate having a fineness of 5 microns, the filtrate was taken to give the active extract.

### Example 2

Chinese medicine composition for eye bags and dark circles fade, preparation APIs are:

11 g of water, 2 g of Ganoderma lucidum extract, ganoderma extract 4 g of black, wheat germ oil 6 g, 3 g of sucrose laurate, chamomile extract 4 g, 4 g of milk protein, 4 g yeast ferment filtrate, dextran 3 grams of sugar, white fungus polysaccharides 3 grams, 3 grams of Angelica root extract, astragalus extract 3 g.

Preparation Method:

(1) in accordance with the amount of raw materials were weighed each drug;

(2) The above composition is immersed in water for 2 hours and heated to 100, incubated 60 minutes;

(3) to said step (2) and (3) respectively for coarse gauze of 250 mesh filter, and the combined filtrate;

(4) The step (3) of the filtrate was cooled to 30, vacuum-suction frame, the degree of vacuum is 0.5 Mpa, the filter plate having a fineness of 5 microns, the filtrate was taken to give the active extract.

Usage: Apply in morning after lotion or cream eye regeneration, Apply on the skin near the eye and shake gently to help shake head penetration.

The present invention quick, seven days up to 70% efficiency, and a certain cure rate, the effective rate of 85% a month. On the poor state of sleep, stress, ultraviolet radiation causes skin dull, no case of allergic reactions, skin irritation and so on.

The following referenced cases are herein incorporated: U.S. Nonprovisional patent application Ser. No. 14/______, filed Dec. 30, 2013 with Attorney Docket No. ShangChen-13-01, and entitled “A traditional Chinese medicine composition that has the effect of soothing and cleaning face”; U.S. Nonprovisional patent application Ser. No. 14/______, filed Dec. 30, 2013 with Attorney Docket No. ShangChen-13-02, and entitled “A Traditional Chinese medicine extract composition with whitening skin care function and its preparation method”; U.S. Nonprovisional patent application Ser. No. 14/______, filed Dec. 30, 2013 with Attorney Docket No. Shangchen-13-03, and entitled “A traditional Chinese medicine composition for pigment repair”; U.S. Nonprovisional patent application Ser. No. 14/______, filed Dec. 30, 2013 with Attorney Docket No. Shangchen-13-04, and entitled “A traditional Chinese medicine composition for repair of acne”; U.S. Nonprovisional patent application Ser. No. 14/______, filed Dec. 30, 2013 with Attorney Docket No. Shangchen-13-05, and entitled “A traditional Chinese medicine composition for anti-wrinkle”; U.S. Nonprovisional patent application Ser. No. 14/______, filed Dec. 30, 2013 with Attorney Docket No. Shangchen-13-07, and entitled “A Chinese medicine composition for nourishing body care and its preparation method”; U.S. Nonprovisional patent application Ser. No. 14/______, filed Dec. 30, 2013 with Attorney Docket No. Shangchen-13-08, and entitled “Chinese medicine composition that having acne cleansing effect”; U.S. Nonprovisional patent application Ser. No. 14/______, filed Dec. 30, 2013 with Attorney Docket No. Shangchen-13-09, and entitled “An anti-aging skin rejuvenation medicine composition and its preparation method”; U.S. Nonprovisional patent application Ser. No. 14/______, filed Dec. 30, 2013 with Attorney Docket No. Shangchen-13-10, and entitled “Chinese medicine composition for pigment desalination”; U.S. Nonprovisional patent application Ser. No. 14/______, filed Dec. 30, 2013 with Attorney Docket No. Shangchen-13-011, and entitled “Chinese medicine composition for acne scar repair”; U.S. Nonprovisional patent application Ser. No. 14/______, filed Dec. 30, 2013 with Attorney Docket No. Shangchen-13-012, and entitled “Chinese medicine composition for Prevent from aging and soothing the skin”; and U.S. Nonprovisional patent application Ser. No. 14/______, filed Dec. 30, 2013 with Attorney Docket No. Shangchen-13-013, and entitled “Chinese medicine composition for Nourishing the skin and its preparation method”, the disclosure of all of which are herein incorporated by reference in its entirety.

What is claimed is:

1. An eye bags and dark circles fade for Chinese medicine composition, wherein said composition comprises the following parts by weight ratio of raw material composition: 10-15 parts water, Ganoderma lucidum extract 2-3 copies, black Ganoderma Extraction 3-4, 5-6 parts wheat germ oil, sucrose laurate 2-3 parts, chamomile extract 3-5 parts, 3-5 parts of milk protein, yeast ferment filtrate 3-5 parts product, dextran 2-3 parts of sugar, white fungus polysaccharides 2-4 copies, angelica root extract 2-3 parts, astragalus extract 2-3 parts.

2. Chinese medicine composition as claimed in claim 1, wherein, wherein the components are: 10-11 parts of water, 2-3 parts of the extract of Ganoderma lucidum, Ganoderma lucidum extract black parts 3-4, 5-6 parts of wheat germ oil, sugar laurate 2-3 parts, chamomile extract 3-4, 3-4 parts of milk protein, the product of yeast fermentation filtrate 3-4, 2-3 glucan copies Tremella 2-3 copies, angelica root 2-3 parts extract, astragalus extract 2-3 parts.

3. Chinese medicine composition as claimed in claim 1, wherein, wherein the components are: 12-13 parts water, 2-3 parts of the extract of Ganoderma lucidum, Ganoderma lucidum extract black parts 3-4, 5-6 parts of wheat germ oil, sugar laurate 2-3 parts, chamomile extract 4-5 parts, 4-5 parts of milk protein, yeast ferment filtrate 4-5 parts product, dextran 2-3 copies, Tremella 3-4, angelica root 2-3 parts extract, astragalus extract 2-3 parts.

4. Chinese medicine composition as claimed in claim 1, wherein the preparation method is: (a) were weighed according to the amount of each raw material; (2) mixing the composition in water and soaked in 0.5 to 2 hours, the above composition total mass and the volume ratio of water (0.5˜1): (10˜30) g/ml, and heated to 80˜100° C., for 0.5 to 1.5 hours; (3) to said step (2) for coarse filtration;
(4) Step (3 filtrate was cooled to 30˜50° C., and vacuum filtration, the filtrate was taken to give the active extract.
Wherein said step (3) of the coarse filter with 200 to 300 mesh gauze filter. Wherein said step (4) is a vacuum suction plate and frame filter, a vacuum degree of 0.05˜0.1 Mpa, the fineness of the filtration plate 1 to 5 microns. The method can be preferably used for preparing the raw extract of the active ingredient in the drug.

5. The medicine composition according to claim 1, wherein the components through a conventional method together with auxiliary materials cream.

6. Chinese medicine composition of claim 1, wherein the cream made from the requirements applicable to its use for the evening after a lotion or cream eye regeneration, Apply on the skin near the eye and shake gently to help shake head penetration.
